(19) United States
(12) Patent Application Publication
(10) Pub. No.: US 2022/0405921 A1
(43) Pub. Date: Dec. 22, 2022

(54) LEUKOCYTE DETECTION METHOD, SYSTEM, ELECTRONIC DEVICE, AND COMPUTER READABLE MEDIUM

(71) Applicant: BOE TECHNOLOGY GROUP CO., LTD., Beijing (CN)

(72) Inventor: Hui DU, Beijing (CN)

(21) Appl. No.: 17/772,657

(22) PCT Filed: Jul. 14, 2021
(86) PCT No.: PCT/CN2021/106221

§ 371 Date: Apr. 28, 2022

(30) Foreign Application Priority Data
Aug. 19, 2020 (CN) 202010837832.4

(51) Int. Cl. G06T 7/00; G06T 7/30; G06V 10/32; G06T 7/13

(52) U.S. Cl. CPC G06T 7/0012; G06T 7/30; G06V 10/32; G06T 7/13; G06T 2207/30104; G06T 2207/30242; G06T 2207/10024

(57) ABSTRACT

Provided are a leukocyte detection method, a system, an electronic device and a computer readable medium. The method comprises: acquiring a microcirculation image (S1); determining a location of an intra-tubular space of a capillary vessel from the microcirculation image (S2); and determining a leukocyte index based on image information of the intra-tubular space of the capillary vessel (S3).

### TECHNICAL FIELD

The present disclosure relates to the technical field of image processing, and in particular a leukocyte detection method, a system, an electronic device and a computer readable medium.

### BACKGROUND

Leukocytes are a very important type of blood cells in human blood. Leukocytes perform many important functions in the human body, such as phagocytosis of foreign matters and production of antibodies, healing of body damages, resistance to invasion by pathogens, immune resistance to diseases, and the like.

Existing leukocyte detection is performed by collecting a peripheral blood (e.g., fingertip blood collection, or earlobe blood collection), which may cause the subject to feel obvious pain. In addition, the blood analysis requires a series of operations such as dilution, sample preparation, counting under a microscope and so on, and manual participation is needed throughout the detection process, rendering the process tedious and time-consuming.

### SUMMARY

The present invention aims to solve at least one of the technical problems in the prior art by providing a leukocyte detection method, a system, an electronic device, and a computer readable medium.

In a first aspect, embodiments of the present disclosure provide a leukocyte detection method comprising:

acquiring a microcirculation image;

determining from the microcirculation image a location of an intra-tubular space of a capillary vessel; and

determining a leukocyte index based on image information of the intra-tubular space of the capillary vessel.

In some embodiments, the step of acquiring a microcirculation image comprises:

acquiring consecutive multi-frame microcirculation images over a predetermined period of time; and

the step of determining a leukocyte index based on the image information of the intra-tubular space of the capillary vessel comprises:

determining a flow amount of leukocytes in the intra-tubular space of the capillary vessel based on the image information of the intra-tubular space of the capillary vessel in the consecutive multi-frame microcirculation images, the flow amount of leukocytes indicating the number of leukocytes passing through an effective cross-section of the capillary vessel per unit time, and the leukocyte index comprising the flow amount of leukocytes.

In some embodiments, the step of determining the flow amount of leukocytes in the intra-tubular space of the capillary vessel based on the image information of the intra-tubular space of the capillary vessel in the consecutive multi-frame microcirculation images comprises:

determining the number of leukocytes passing through a detection region over the predetermined period of time, based on color change in the detection region of the intra-tubular space of the capillary vessel in the consecutive multi-frame microcirculation images, and

determining the flow amount of leukocytes based on the predetermined period of time and the number of leukocytes passing through the detection region over the predetermined period of time.

In some embodiments, the step of determining the number of leukocytes passing through the detection region over the predetermined period of time, based on color change in the detection region of the intra-tubular space of the capillary vessel in the consecutive multi-frame microcirculation images comprises:

performing an energy analysis of the detection region in the consecutive multi-frame microcirculation images to obtain an energy spectrum corresponding to the detection region; and

counting the number of energy peaks in the energy spectrum as the number of leukocytes passing through the detection region over the predetermined period of time.

In some embodiments, after the step of determining a flow amount of leukocytes in the intra-tubular space of the capillary vessel, the method further comprises:

assessing, on the basis of the flow amount of leukocytes, a distribution density of leukocytes in a blood; and

the leukocyte index comprises the distribution density of leukocytes.

In some embodiments, prior to the step of acquiring a microcirculation image, the method further comprises:

tuning system parameters of a shooting system based on a preset baseline colorimetric card.

In some embodiments, the system parameters include at least one of saturation, exposure and color difference.

In some embodiments, after the step of acquiring a microcirculation image and prior to the step of determining from the microcirculation image a location of an intra-tubular space of the capillary vessel, the method further comprises:

normalizing and aligning the microcirculation image.

In some embodiments, after the step of normalizing and aligning the microcirculation image and before determining from the microcirculation image a location of an intra-tubular space of the capillary vessel, the method further comprises:

binarizing the microcirculation image after subjected to the normalization and alignment processing.

In some embodiments, the step of determining from the microcirculation image a location of an intra-tubular space of the capillary vessel comprises:

determining, by means of an edge detection algorithm, an edge of the capillary vessel in the microcirculation image; and

determining the location of the intra-tubular space of capillary vessels based on the edge detection results of the capillary vessel.

In some embodiments, the edge detection algorithm comprises: Laplacian of Gaussian edge detection algorithm.

In some embodiments, after the step of determining, by means of an edge detection algorithm, an edge of the capillary vessel in the microcirculation image and before the step of determining the location of the intra-tubular space of capillary vessels based on the edge detection results of the capillary vessel, the method further comprises:

enhancing and extracting the edges of the capillary vessel by a maximum interclass variance method.

In a second aspect, embodiments of the present disclosure further provide a leukocyte detection system, comprising:

an image acquisition module configured to acquire a microcirculation image;

a location determination module configured to determine from the microcirculation image a location of an intra-tubular space of the capillary vessel; and

an index determination module configured to determine a leukocyte index based on image information of the intra-tubular space of the capillary vessel.

In a third aspect, embodiments of the present disclosure further provide an electronic device comprising:

one or more processors;

a memory having one or more programs stored thereon, which, when the one or more programs is/are executed by the one or more processors, causes the one or more processors to implement the leukocyte detection method as provided in the first aspect.

In a fourth aspect, embodiments of the present disclosure further provide a computer readable medium having a computer program stored thereon, wherein when the program is executed by the processor, the leukocyte detection method as provided in the first aspect is implemented.

### BRIEF DESCRIPTION OF DRAWINGS

FIG. 1 is a flow diagram of a leukocyte detection method provided by embodiments of the present disclosure.

FIG. 2 is a schematic diagram of a microcirculation image at a partial location on the nail wall in embodiments of the present disclosure.

FIG. 3 is a flowchart of a specific implementation of the step S2 in embodiments of the present disclosure.

FIG. 4 is a schematic diagram of the capillary vessel in the microcirculation image after subjected to the edge recognition in the embodiments of the present disclosure.

FIG. 5 is a flowchart of another specific implementation of the step S2 in embodiments of the present disclosure.

FIG. 6 is a flowchart of another leukocyte detection method provided by embodiments of the present disclosure.

FIG. 7 is a flowchart of yet another leukocyte detection method provided in embodiments of the present disclosure.

FIG. 8 is a flowchart of still another leukocyte detection method provided by embodiments of the present disclosure.

FIG. 9 is a flowchart of a specific implementation of the step S3 in embodiments of the present disclosure.

FIG. 10 is a flowchart of a specific implementation of the step S301 in embodiments of the present disclosure.

FIG. 11 is a flowchart of a further leukocyte detection method provided by embodiments of the present disclosure.

FIG. 12 is a block diagram of a structure of a leukocyte detection system provided in embodiments of the present disclosure.

### DETAILED DESCRIPTION OF EMBODIMENTS

To enable those skilled in the art to better understand the technical embodiments of the present invention, a leukocyte detection method, a system, an electronic device, and a computer readable medium provided by the present invention are described in detail below in conjunction with the accompanying drawings.

Illustrative embodiments will be described more fully below with reference to the accompanying drawings, but said illustrative embodiments may be embodied in different forms and should not be construed as being limited to the embodiments set forth herein. Instead, these embodiments are provided for the purpose of making the present disclosure thorough and complete and will enable those skilled in the art to fully understand the scope of the present disclosure.

In the absence of conflict, the various embodiments of the present disclosure and the various features in the embodiments may be combined with each other.

As used herein, the term “and/or” includes any and all combinations of one or more related enumerated entries.

The terms used herein are used only to describe particular embodiments and are not intended to limit the present disclosure. As used herein, the singular forms “a” and “the” are also intended to include the plural forms, unless the context clearly indicates otherwise. It will also be understood that when the terms “comprising”, “comprise(s)”, “including”, “include(s)” and/or “made from . . . ” are used in this specification, they designate the presence of said feature, whole, step, operation, element, and/or assembly but do not exclude the presence or addition of one or more other features, wholes, steps, operations, elements, assemblies, and/or groups thereof.

Unless otherwise defined, all terms used herein (including technical and scientific terms) have the same meaning as commonly understood by those of ordinary skill in the art. It will also be understood that the terms such as those defined in commonly used dictionaries should be construed to have a meaning consistent with their meaning in the relevant art and in the context of the present disclosure, and will not be construed to have an idealized or overly formal meaning unless expressly so defined herein.

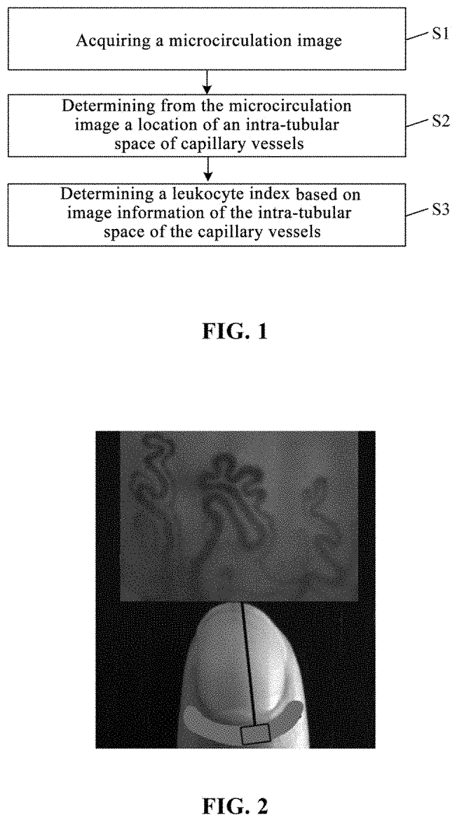

FIG. 1 is a flow diagram of a leukocyte detection method provided in embodiments of the present disclosure. As shown in FIG. 1, the leukocyte detection method is based on a leukocyte detection system, and the leukocyte detection method comprises steps S1 to S3.

Step S1 is acquiring a microcirculation image.

In embodiments of the present disclosure, where the object of detection is a human body, for example, the microcirculation image at certain locations of the human body can be captured by a shooting system, and then the shooting system sends the captured image to the leukocyte detection system for subsequent processing by the leukocyte detection system.

In some embodiments, the shooting system is a shooting system of a cell phone, specifically including a camera (a hardware device) and an image generation system (software system). The camera is used to take a picture and output a corresponding induction signal, and the image generation system is used to generate a corresponding image based on the induction signal output by the camera.

In the embodiments of the present disclosure, where the object of detection is a human body, for example, there are more than a dozen of locations of the human body which can be used to observe microcirculation, but the locations which are the most commonly used and representative of the microcirculation status of the whole body are the nail wall (the raised skin base layer at the nail groove) and the conjunctiva of the eyeball, of which the nail wall is the best window to observe the microcirculation of the human body. In an embodiment of the present disclosure, the microcirculation image of the human nail wall is captured as an example.

In order to clearly capture the microcirculation image of the nail wall, magnification processing (generally 30 times or more) is required during the filming process, and currently the optical zoom and digital zoom can be used to achieve magnification. Among them, the digital zoom magnification will make the generated image have some information damage, so the optical zoom is preferred for magnification. Specifically, if the camera equipped on the cell phone itself has the optical zoom function and the optical zoom magnification is above 30 times, the optical zoom magnification of the camera may be directly adjusted, and then a picture of the nail wall is captured. If the camera equipped on the cell phone itself does not have the optical zoom function or has the optical zoom function but the maximum optical zoom magnification is less than 30 times, the camera may be configured with an additional magnifying glass at the light inlet of the camera, so that the optical zoom magnification of the camera combined with the magnifying glass is greater than 30.

FIG. 2 shows a schematic diagram of the microcirculation image at the partial location of the nail wall in the embodiments of the present disclosure. As shown in FIG. 2, the finger nail wall is a skin fold covering the root of the nail, and its epidermis is a stratified squamous epithelium, with dermal papillae formed by connective tissue protrusions under the epithelium. Generally, there is one capillary vessel in each papilla, which extends to the epidermis and becomes flush with the epidermis when approaching the epidermis. The image of microcirculation in the nail wall can be clearly captured at an optical zoom magnification of 30×, with the microcirculation image containing a clear capillary image.

In step S2, the location of the intra-tubular space of the capillary vessel is determined from the microcirculation image.

In step S2, the location of the intra-tubular space of the capillary vessel can be determined from the microcirculation image by image processing techniques.

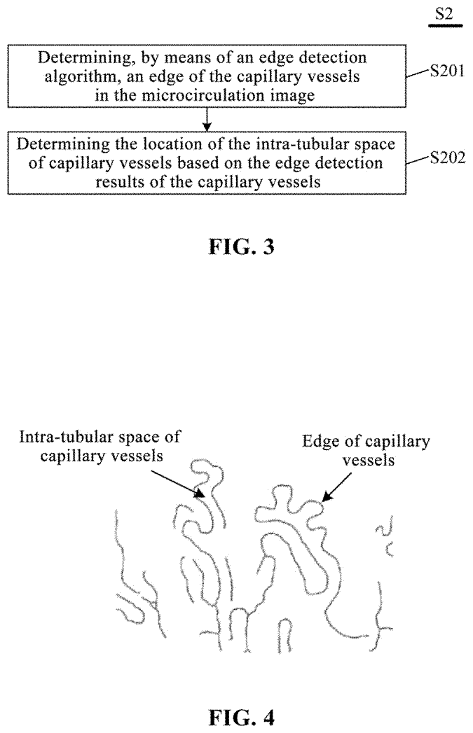

FIG. 3 is a flow chart of one specific implementation of step S2 in embodiments of the present disclosure. As shown in FIG. 3, in some embodiments, the step S2 comprises steps S201 and S202. Step S201 is determining an edge of the capillary vessel in the microcirculation image by an edge detection algorithm.

FIG. 4 shows a schematic diagram of the capillary vessel in the microcirculation image after edge identification in the embodiments of the present disclosure. As shown in FIG. 4, a clear outline of the capillary vessel is obtained using an edge detection algorithm to identify the edges of the capillary vessel in the microcirculation image.

In some embodiments, the edge detection algorithm includes the Laplacian of Gaussian (LOG) edge detection algorithm, which is formed by combining a Gaussian operator and a Laplacian operator. Specifically, the image is first smoothed using the Gaussian operator, and then the edges of the image are detected by the Laplacian operator using zero-crossing of the second order differential. The specific operation process belongs to the conventional techniques in the field and will not be detailedly described here.

In some embodiments, the LOG edge detection algorithm can be represented by the following equation:

$$LOG ( x , y ) = - \frac{1}{2 π {σ}^{4}} [ 2 - \frac{{x}^{2} + {y}^{2}}{{σ}^{2}} ] exp ( - \frac{{x}^{2} + {y}^{2}}{2 {σ}^{2}} )$$

LOG(x,y) denotes the result of the LOG edge detection operation on the coordinates (x, y), and σ is a width of the Gaussian kernel. During actual tests, it has been found that the edge detection effect is better when the value of σ is 1.4. Of course, the value of σ may be set and adjusted according to practical needs.

Step S202 is determining the location of the intra-tubular space of the capillary vessel based on the edge detection results of the capillary vessel.

In step S202, the location of the intra-tubular space of the capillary vessel can be determined based on the edge detection results of the capillary vessel.

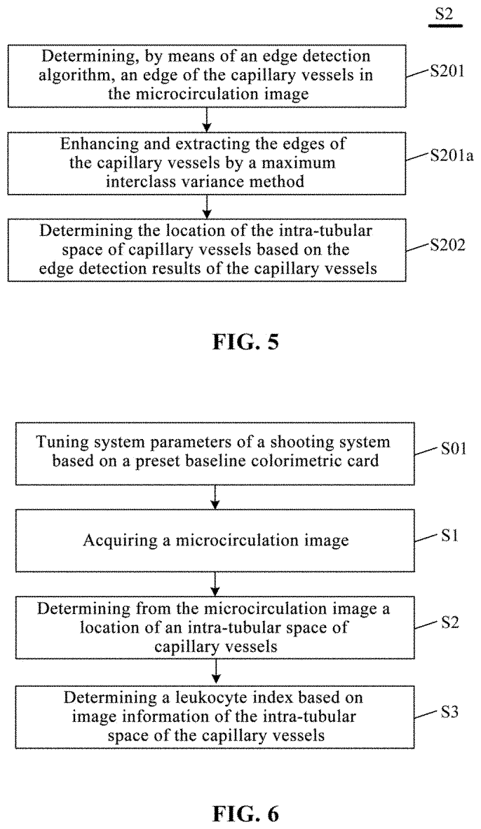

FIG. 5 is a flow chart of another specific implementation of the step S2 in embodiments of the present disclosure. As shown in FIG. 5, in some embodiments, the step S2 includes not only step S201 and step S202 in the above embodiment, but also step S201a between steps S201 and S202. Only step S201a is described in detail below.

S201a is enhancing and extracting the edges of the capillary vessel by a maximum interclass variance method.

In the case shown in FIG. 4, after the processing of the LOG edge detection algorithm, the maximum interclass variance method is adopted to further process the microcirculation image, such that the edge information of the capillary vessel can be enhanced and extracted to facilitate the accurate determination of the location of the intra-tubular space of the capillary vessel in the subsequent process.

Step S3 is determining the leukocyte index based on the image information of the intra-tubular space of the capillary vessel.

In step S3, the image of the intra-tubular space of the capillary vessel is processed based on the image processing technique to obtain the leukocyte information of the intra-tubular space, such as the number of leukocytes, the location of leukocyte distribution, the flow of leukocytes, and other information. Exemplarily, the deep learning technique is used to identify the leukocytes in the intra-tubular space, so that the number and location of leukocytes contained in the intra-tubular space of the capillary vessel in each frame of the microcirculation image can be obtained. The flow amount of leukocytes can be obtained based on the change of leukocytes in the intra-tubular space of capillary vessels in multiple frames of microcirculation image.

Based on the leukocyte information in the intra-tubular space of capillary vessels, the leukocyte index of the human body can be evaluated. Among others, the leukocyte index can be used to assess whether the leukocyte status in the human body is abnormal. Exemplarily, the leukocyte index may include at least one of total leukocyte count, the flow amount of leukocytes, and the distribution density of leukocytes.

In embodiments of the present disclosure, by acquiring a microcirculation image and processing the microcirculation image based on image processing techniques, the status of the leukocytes in the intra-tubular space of the capillary vessel can be acquired, and thus the leukocyte index can be obtained. In the above-mentioned detection process, there is no need for blood collection and the user will no longer feel pain; at the same time, the whole testing process does not require manual participation and takes a short time.

FIG. 6 shows a flow chart of another leukocyte detection method provided in embodiments of the present disclosure. As shown in FIG. 6, the detection method includes not only the above steps S1 to S3, but also step S01 before the step S1. Only step S01 is described in detail below.

Step S01 is tuning the system parameters of a shooting system based on a preset baseline colorimetric card.

Depending on the camera performances of different cell phones and the capturing environments, the saturation and exposure degree of the microcirculation image captured by different users at different moments are different and there are color differences. As a result, it will cause interference to the subsequent image processing, thus affecting the final detection results.

In order to solve the above technical problem, a special colorimetric card (i.e., preset baseline colorimetric card) is introduced in the embodiments of the present disclosure, and the user needs to shoot the preset baseline colorimetric card first and send the generated image of the colorimetric card to the leukocyte detection system before capturing the microcirculation image through the shooting system. The leukocyte detection system compares the color characteristics of the colorimetric card image received with those pre-stored for the preset baseline colorimetric card, and tunes the system parameters according to the differences in the color characteristics between them to ensure that the color characteristics of the microcirculation patterns taken by different users (different shooting systems) in different environments are substantially or completely the same, such that the accuracy of the detection results can be improved.

In some embodiments, the system parameters include at least one of saturation, exposure, and color difference.

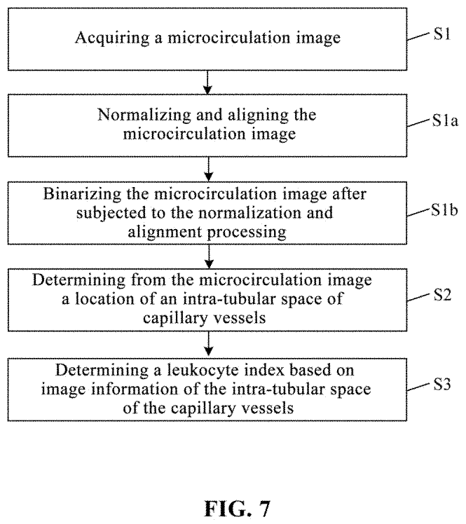

FIG. 7 is a flow chart of yet another leukocyte detection method provided by embodiments of the present disclosure. As shown in FIG. 7, the detection method includes not only steps S1 to S3 as described above, but also step S1a and step S1b between the steps S1 and S2. Step S1a is described in detail below.

Step S1a is normalizing and aligning the microcirculation image.

Here, the image normalization refers to the process of transforming an image into a fixed standard form by a series of standard processing and transformations, and there is no particular limit to the specific normalization algorithm used in the technical solutions of the present disclosure. The image alignment process is completed using the grayscale information method, which is mainly used to determine the geometric position distribution of the image and facilitate the identification of the corresponding information area.

Step S1b is performing the binarization process on the microcirculation image after completing the normalization and alignment processes.

The binarization process of the image is to form the image into two gray values of 0 and 255 to facilitate the identification of capillary vessels later.

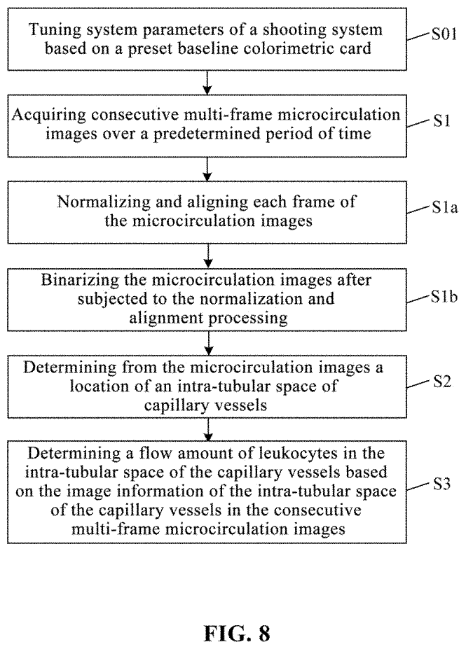

FIG. 8 is a flow chart of a further leukocyte detection method provided by embodiments of the present disclosure. As shown in FIG. 8, the method comprises steps S01 to S3.

Step S01 is tuning the system parameters of the shooting system based on a preset baseline colorimetric card.

Step S1 is acquiring continuous (consecutive) multi-frame microcirculation images over a predetermined time duration.

In this embodiment, the continuous multi-frame microcirculation images can be acquired by video recording over a period of time (which may be set by the leukocyte detection system or decided by the user).

The user may capture the image of the nail wall through the shooting system, where the magnification of the shooting system is 30× or more, and the distance between the camera and the finger during the shooting process is about 5 cm, so that the shooting system can capture the microcirculation image of the nail wall.

Step S1a is normalizing and aligning each frame of the microcirculation images.

Step S1b is performing the binarization process on the microcirculation image after completing the normalization and alignment processes.

Step S2 is determining the location of the intra-tubular space of the capillary vessel from the microcirculation image.

Step S3 is determining the flow amount of leukocytes in the intra-tubular space of the capillary vessel based on the image information of the intra-tubular space of the capillary vessel in the consecutive multi-frame microcirculation images.

Here, the flow amount of leukocytes indicates the number of leukocytes flowing through the effective cross-section of the capillary vessel per unit time, and the leukocyte index includes the flow amount of leukocytes.

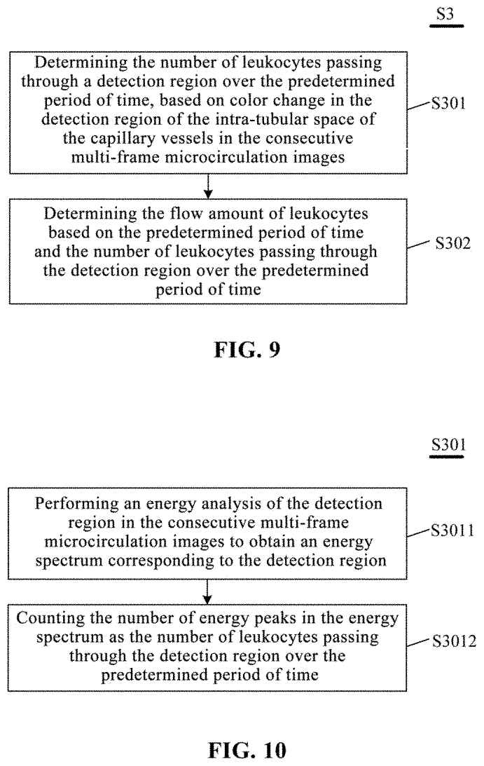

FIG. 9 is a flow diagram of one specific implementation of the step S3 in the embodiments of the present disclosure. As shown in FIG. 9, in some embodiments the step S3 comprises steps S301 and S302.

Step S301 is determining the number of leukocytes passing through the detection region within a predetermined time, based on color change in the detection region of the intra-tubular space of the capillary vessel in the consecutive multi-frame microcirculation images.

In some embodiments, the microcirculation image contains images of the intra-tubular space of multiple capillary vessels. To facilitate detection, one of the capillary vessel is used as the detection object, and the area in which the capillary vessel is located is taken as the detection area.

Leukocytes are large in size and can pass through capillary vessels only one by one. When the leukocytes pass through the capillary vessels, the color of the intra-tubular space of the capillary vessel changes, and the number of leukocytes passing through the detection area can be counted based on this color change.

FIG. 10 is a flow chart of one specific implementation of step S301 in embodiments of the present disclosure. As shown in FIG. 10, the step 301 comprises:

Step S3011, performing energy analysis of the detection region in the consecutive multi-frame microcirculation images to obtain the energy spectrum corresponding to the detection region; and

Step S3012, counting the number of energy peaks in the energy spectrum as the number of leukocytes passing through the detection region during a predetermined time period.

As the leukocytes pass through the capillary vessels, the color of the intra-tubular space of the capillary vessel changes, but the color change is very weak. For this reason, in an embodiment of the present disclosure, the energy analysis is performed on the detection area in the continuous multi-frame microcirculation images to generate an energy spectrum, which can effectively reflect the weak change in color of the detection area. An energy peak will appear in the energy spectrum when leukocytes pass the detection area, and the number of leukocytes passing through the detection area within a predetermined time period can be obtained by counting the number of energy peaks in the energy spectrum of the detection area within the predetermined time period.

Step S302 is determining the flow amount of leukocytes based on the predetermined time and the number of leukocytes passing through the detection area during the predetermined time period.

In step S302, the flow amount of leukocytes in the capillary vessels is obtained by dividing the number of leukocytes counted in step S302 by the predetermined time. Since the magnitude of the flow amount of leukocytes itself reflects the condition of leukocytes in the blood, the flow amount of leukocytes itself can be used as a leukocyte index.

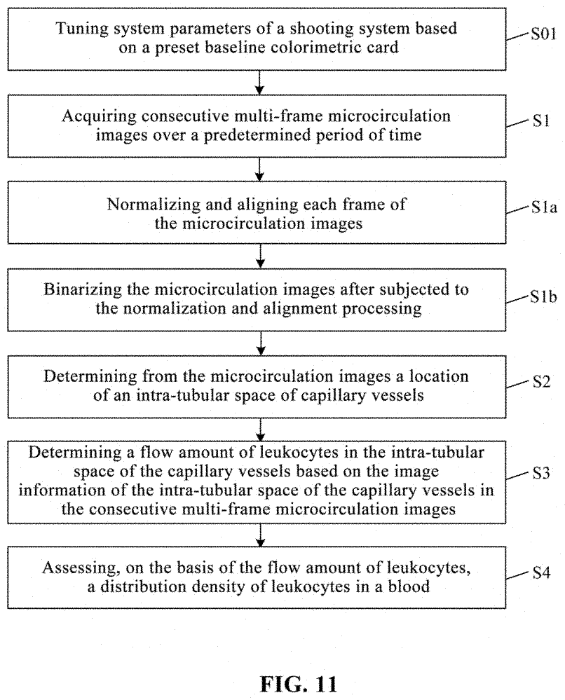

FIG. 11 shows a flow chart of a further leukocyte detection method provided by embodiments of the present disclosure. As shown in FIG. 11, the embodiment provides a leukocyte detection method based on the method shown in FIG. 10 above, but further includes step S4 after step S3. Only step S4 is described in detail below.

Step S4 is assessing the distribution density of leukocytes in the blood based on the flow amount of leukocytes.

Here, the larger the flow amount of leukocytes calculated in step S3, the more the total number of leukocytes in the blood and the higher the distribution density of leukocytes; or the smaller the flow amount of leukocytes calculated in step S3, the less the total number of leukocytes in the blood and the lower the distribution density of leukocytes.

The mapping relationship between the flow amount of leukocytes and the total number and the distribution density of leukocytes can be determined by a preliminary experiment. In the step S4, the total number and the distribution density of leukocytes in the blood may be assessed based on the pre-obtained mapping relationship and the flow amount of leukocytes obtained in the step S3.

In some embodiments, the leukocyte index may include the total number of leukocytes and/or the distribution density of leukocytes.

The present disclosure provides a leukocyte detection method in which a leukocyte index can be obtained by acquiring microcirculation image and processing the microcirculation image based on image processing techniques to obtain the status of leukocytes in the intra-tubular space of capillary vessels. In the above-described detection processes, there is no need for blood collection and the user will no longer feel pain; at the same time, the whole detection process does not require manual participation and takes a short time.

It should be noted that the different steps in the above-described embodiments of the leukocyte detection method can be combined with each other, and the new technical solutions obtained through the combination should also fall within the scope of protection of the present disclosure.

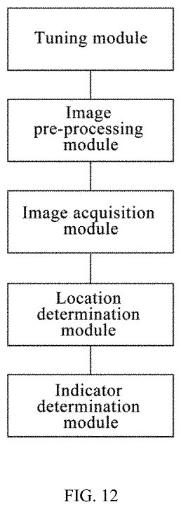

FIG. 12 shows a block diagram of the structure of a leukocyte detection system provided in embodiments of the present disclosure. As shown in FIG. 12, the leukocyte detection system includes: an image acquisition module, a location determination module and an index determination module.

The image acquisition module is configured to acquire a microcirculation image; the location determination module is configured to determine the location of the intra-capillary space of capillary vessels from the microcirculation image; and the index determination module is configured to determine a leukocyte index based on the image information of the intra-capillary space of capillary vessels.

In some embodiments, the leukocyte detection system further comprises a tuning module configured to tune the system parameters of the shooting system based on a predetermined baseline colorimetric card.

In some embodiments, the leukocyte detection system further comprises an image pre-processing module configured to normalize and align the microcirculation image. Further, the image pre-processing module is configured to binarize the microcirculation image after subjected to the normalization and alignment processes.

The leukocyte detection systems provided by the embodiments of the present disclosure can be used to implement the leukocyte detection method provided in any of the preceding embodiments, and the specific description of each functional module can be found in the corresponding content of the preceding embodiments and will not be repeated here.

Embodiments of the present disclosure also provide an electronic device comprising one or more processors and a memory; wherein the memory has one or more programs stored thereon, which, when the one or more programs is/are executed by the one or more processors, causes the one or more processors to implement the leukocyte detection method provided in any of the preceding embodiments. That is, the program corresponding to the leukocyte detection system is installed on the electronic device.

In some embodiments, the shooting system may exist independently of the electronic device, and the shooting system sends the captured microcirculation image to the leukocyte detection system via wired or wireless means for processing by the leukocyte detection system.

In some embodiments, the shooting system may be integrated into the electronic device. In this case, the electronic device may be a structure or device that has a shooting function and a data processing function, including cell phones, tablets, cameras, etc.

Embodiments of the present disclosure also provide a computer readable medium having a computer program stored thereon, wherein when the program is executed by a processor, the leukocyte detection method provided in any of the previous embodiments is implemented.

It will be understood by those of ordinary skill in the art that all or some of the steps, systems, and functional modules/units of the devices in the methods disclosed above may be implemented as software, firmware, hardware, and suitable combinations thereof. In a hardware implementation, the division of functional modules/units mentioned in the above description does not necessarily correspond to a division of physical components. For example, a physical component may have multiple functions, or one function or step may be performed cooperatively by several physical components. Some or all of the physical components may be implemented as software executed by a processor, such as a central processing unit, a digital signal processor, or a microprocessor, or be implemented as hardware, or be implemented as an integrated circuit, such as a specialized integrated circuit. Such software may be distributed on a computer-readable medium, which may include computer storage media (or non-transitory media) and communication media (or transitory media). As is well known to those of ordinary skill in the art, the term “computer storage medium” includes volatile and non-volatile, removable and non-removable media implemented in any method or technology for storing information, such as computer readable instructions, data structures, program modules, or other data. Computer storage media include, but are not limited to, RAM, ROM, EEPROM, flash memory or other memory technology, CD-ROM, digital versatile disk (DVD) or other optical disk storage, magnetic cartridges, magnetic tapes, disk storage or other magnetic storage devices, or any other media that can be used to store desired information and can be accessed by a computer. In addition, it is well known to those of ordinary skill in the art that the communication media typically contain computer-readable instructions, data structures, program modules, or other data in modulated data signals such as carrier waves or other transmission mechanisms, and may include any information delivery medium.

Exemplary embodiments have been disclosed herein, and while specific terms are employed, they are used and should be construed only in a general illustrative sense and are not intended to be limiting. In some examples, it will be apparent to those skilled in the art that features, characteristics and/or elements described with reference to particular embodiments may be used alone, or may be used in combination with features, characteristics and/or elements described with reference to other embodiments, unless otherwise expressly indicated. Thus, it will be understood by those of skill in the art that various changes in form and detail may be made without departing from the scope of the present disclosure as set forth by the appended claims.

1. A leukocyte detection method, comprising:
acquiring a microcirculation image,
determining from the microcirculation image a location of an intra-tubular space of a capillary vessel, and
determining a leukocyte index based on image information of the intra-tubular space of the capillary vessel.

2. The leukocyte detection method according to claim 1, wherein the step of acquiring a microcirculation image comprises:
acquiring consecutive multi-frame microcirculation images over a predetermined period of time; and
the step of determining a leukocyte index based on the image information of the intra-tubular space of the capillary vessel comprises:
determining a flow amount of leukocytes in the intra-tubular space of the capillary vessel based on the image information of the intra-tubular space of the capillary vessel in the consecutive multi-frame microcirculation images, the flow amount of leukocytes indicating the number of leukocytes passing through an effective cross-section of the capillary vessel per unit time, and the leukocyte index comprising the flow amount of leukocytes.

3. The leukocyte detection method according to claim 2, wherein the step of determining the flow amount of leukocytes in the intra-tubular space of the capillary vessel based on the image information of the intra-tubular space of the capillary vessel in the consecutive multi-frame microcirculation images comprises:
determining the number of leukocytes passing through a detection region over the predetermined period of time, based on color change in the detection region of the intra-tubular space of the capillary vessel in the consecutive multi-frame microcirculation images, and
determining the flow amount of leukocytes based on the predetermined period of time and the number of leukocytes passing through the detection region over the predetermined period of time.

4. The leukocyte detection method according to claim 3, wherein the step of determining the number of leukocytes passing through the detection region over the predetermined period of time based on color change in the detection region of the intra-tubular space of the capillary vessel in the consecutive multi-frame microcirculation images comprises:
performing an energy analysis of the detection region in the consecutive multi-frame microcirculation images to obtain an energy spectrum corresponding to the detection region; and
counting the number of energy peaks in the energy spectrum as the number of leukocytes passing through the detection region over the predetermined period of time.

5. The leukocyte detection method according to claim 2, wherein, after the step of determining a flow amount of leukocytes in the intra-tubular space of the capillary vessel, the method further comprises:
assessing, on the basis of the flow amount of leukocytes, a distribution density of leukocytes in a blood;
wherein the leukocyte index comprises the distribution density of leukocytes.

6. The leukocyte detection method according to claim 1, wherein, prior to the step of acquiring a microcirculation image, the method further comprises:
tuning system parameters of a shooting system based on a preset baseline colorimetric card.

7. The leukocyte detection method according to claim 6, wherein the system parameters include at least one of saturation, exposure and color difference.

8. The leukocyte detection method according to claim 1, wherein, after the step of acquiring a microcirculation image and prior to the step of determining from the microcirculation image a location of an intra-tubular space of the capillary vessel, the method further comprises:
normalizing and aligning the microcirculation image.

9. The leukocyte detection method according to claim 8, wherein, after the step of normalizing and aligning the microcirculation image and before determining from the microcirculation image a location of an intra-tubular space of the capillary vessel, the method further comprises:
binarizing the microcirculation image after subjected to the normalization and alignment processing.

10. The leukocyte detection method according to claim 1, wherein the step of determining from the microcirculation image a location of an intra-tubular space of capillary vessels comprises:
determining, by means of an edge detection algorithm, an edge of the capillary vessel in the microcirculation image; and
determining the location of the intra-tubular space of the capillary vessel based on the edge detection results of the capillary vessel.

11. The leukocyte detection method according to claim 10, wherein the edge detection algorithm comprises a Laplacian of Gaussian edge detection algorithm.

12. The leukocyte detection method according to claim 10, wherein, after the step of determining, by means of an edge detection algorithm, an edge of the capillary vessel in the microcirculation image and before the step of determining the location of the intra-tubular space of capillary vessels based on the edge detection results of the capillary vessel, the method further comprises:
enhancing and extracting the edge of the capillary vessel by a maximum interclass variance method.

13. A leukocyte detection system, comprising:
an image acquisition module configured to acquire a microcirculation image;
a location determination module configured to determine from the microcirculation image a location of an intra-tubular space of a capillary vessel; and
an index determination module configured to determine a leukocyte index based on image information of the intra-tubular space of the capillary vessel.

14. An electronic device, comprising:
one or more processors,
a memory having one or more programs stored thereon, which, when the one or more programs is/are executed by the one or more processors, causes the one or more processors to implement the method of claim 1.

15. A computer readable medium having a computer program stored thereon, wherein when the program is executed by a processor, the method of claim 1 is implemented.

16. An electronic device, comprising:
one or more processors,
a memory having one or more programs stored thereon, which, when the one or more programs is/are executed by the one or more processors, causes the one or more processors to implement the method of claim 2.

17. An electronic device, comprising:
one or more processors,
a memory having one or more programs stored thereon, which, when the one or more programs is/are executed by the one or more processors, causes the one or more processors to implement the method of claim 3.

18. An electronic device, comprising:
one or more processors,
a memory having one or more programs stored thereon, which, when the one or more programs is/are executed by the one or more processors, causes the one or more processors to implement the method of claim 4.

19. An electronic device, comprising:
one or more processors,
a memory having one or more programs stored thereon, which, when the one or more programs is/are executed by the one or more processors, causes the one or more processors to implement the method of claim 5.

20. An electronic device, comprising:
one or more processors,
a memory having one or more programs stored thereon, which, when the one or more programs is/are executed by the one or more processors, causes the one or more processors to implement the method of claim 6.
